(19) United States
(12) Patent Application Publication
(10) Pub. No.: US 2014/0058241 A1
(43) Pub. Date: Feb. 27, 2014

(54) Method and Apparatus for Assessing Neurocognitive Status

(71) Applicants: Ross Apparies, New Hope, PA (US); Mark Pflieger, La Mesa, CA (US)

(72) Inventors: Ross Apparies, New Hope, PA (US); Mark Pflieger, La Mesa, CA (US)

(21) Appl. No.: 13/972,603

(22) Filed: Aug. 21, 2013

Related U.S. Application Data
(60) Provisional application No. 61/691,875, filed on Aug. 22, 2012.

(51) Int. Cl. A61B 5/0484; A61B 5/00; A61B 5/0478; A61B 5/04; A61B 3/113

(52) U.S. Cl. CPC A61B 5/04842; A61B 5/04012; A61B 3/113; A61B 5/0478; A61B 5/0006
USPC 600/383; 600/544

(57) ABSTRACT

A method and apparatus for neurological assessment are provided. More specifically, the present invention relates to assessing central nervous system (CNS) function or the change in CNS function. The system (10) includes a monitoring device (20) for monitoring the brain activity of a patient (5) in response to one or more stimuli provided to the patient. Data from the monitoring device (20) is analyzed to determine a measure of CNS condition for the patient. The patient data can be compared with similar data obtained for the patient at a different time to aid a medical professional in the diagnosis or treatment of a neurological or neurodegenerative condition.

### PRIORITY CLAIM

The present applicant claims priority to U.S. Provisional Application No. 61/691,875 filed on Aug. 22, 2012. The entire disclosure of the foregoing application is incorporated herein by reference.

### FIELD OF THE INVENTION

The present invention relates to the field of neurological assessment. More specifically, the present invention relates to assessing neurocognitive status based on electrical monitoring of brain activity in response to particular stimuli. More specifically, the present invention relates to assessing central nervous system (CNS) function or the change in CNS function.

### BACKGROUND

Assessing cognitive functioning can be critical in assessing patient treatment and care. Various conditions from concussions to Alzheimer's disease affect a patient's cognitive functioning. However, it can be difficult to accurately assess a patient's cognitive functioning to evaluate whether a patient has such a condition and/or whether the condition has improved or degraded.

Numerous technologies have been developed for monitoring brain activity, including electroencephalography (EEG), functional magnetic resonance imaging (fMRI), magnetoencephalography (MEG) and others. Despite the availability of these technologies, neurocognitive or CNS function is still predominantly evaluated by subjective report. In addition to being unreliable, subjective reporting lacks sufficient quantification to make it a desirable assessment methodology.

More recently, several methodologies have been proposed which use one or more brain monitoring technologies in an attempt to assess changes in CNS functioning. A significant shortcoming is that the known systems use an approach founded on normative comparisons between a patient's data and a reference group. Such an approach has several shortcomings. For instance, such an approach requires collection of a significant amount of normative data prior to being able to assess the performance of an individual statistically. This can be time consuming even if norms are only collected by sex and by age, both of which can be critical variables. Additionally, the results address only where an individual lies relative to the performance of a normative population as characterized statistically by a normal (Gaussian) distribution. By definition, in a normal distribution some individuals will lie more than 3 standard deviations above or below the mean. This normative data base approach fails to address the critical issue that an individual's most appropriate metric of comparison is that individual. Thus, an individual who is “normally” 3 standard deviations above the mean who has an “event” resulting in cognitive degradation leading to an evaluation placing the individual 0.5 standard deviations above the mean would still be considered normal. However, when compared only to one's self, the change of 2.5 standard deviations is significant.

### SUMMARY OF THE INVENTION

In light of the shortcomings of the prior art, the present invention provides a system for accurately assessing cognitive or CNS change in a patient based on objective criteria. According to one aspect, the invention provides a method for assessing CNS change. The method includes the steps of providing a patient with a first set of stimuli that evokes a series of CNS responses. The responses are measured and processed to create a first set of CNS data for the patient. The data may be processed in a variety of methodologies. For instance, the CNS data may be processed to assess a measure of cortical connectivity for the data. Additionally, the data may be compared with a second set of CNS data for the patient and a measure of CNS variation may be calculated based on the comparison.

According to another aspect, the present invention provides an apparatus for assessing cognitive or CNS change. The apparatus comprises a stimulus generator, a brain function monitor and a processor for processing data from the monitor. The stimulus generator is configured to provide a set of stimuli to a patient to evoke a series of neurocognitive or CNS responses. The monitor is configured to monitor the patient's brain activity and provide data corresponding to the series of neurocognitive or CNS responses. The processor is configured to process the data from the monitor to create a first data set representing the patient's responses to the first set of stimuli at a first point in time. According to one aspect, the processor is configured to assess a cortical connectivity measure based on the data from the brain function monitor and to compare the data set with a second data set representing the patient's responses to stimuli from the stimuli generator at a second point in time and calculate a measure of cognitive or CNS variance in response to the comparison of the patients first and second data sets.

Another aspect of the present invention includes a method for assessing cognitive change. The method includes the step of providing a first set of stimuli to a patient to evoke a series of central nervous system responses. A series of central nervous system responses are measured by monitoring the patient's brain activity and providing data corresponding to the series of central nervous system responses. The data is processed to create a first data set representing the patient's responses to the first set of stimuli at a first point in time. The processing may comprise assessing a cortical connectivity measure. The first data set is compared with a second data set representing the patient's responses to stimuli at a second point in time. A measurement of variation is calculated in response to the step of comparing the patient's first and second cognitive data sets.

Yet another aspect of the present invention is an apparatus for assessing a change in central nervous system function. The apparatus includes a stimuli generator operable to provide a first set of stimuli to a patient to evoke a series of central nervous system responses. A brain function monitor is operable to monitor the patient's brain activity and provide data corresponding to the series of central nervous system responses. A processor is configured to process the data from the brain function monitor to create a first data set representing the patient's responses to the first set of stimuli at a first point in time. The processor may assess a cortical connectivity measure. Additionally, the processor is configured to compare the first data set with a second data set representing the patient's responses to stimuli from the stimuli generator at a second point in time and calculate a measurement of central nervous system variation in response to the comparison of the patient's first and second data sets. The measurement of central nervous system variation is based on an assessment of a cortical connectivity measure based on the data from the brain function monitor.

A further aspect of the present invention is an apparatus having means for providing a first set of stimuli to a patient to evoke a series of central nervous system responses. A means for monitoring brain activity provides data corresponding to the series of central nervous system responses. A means for processing the data from the means for monitoring brain activity creates a first data set representing the patient's responses to the first set of stimuli at a first point in time. The processor may assess a cortical connectivity measure. The means for processing compares the first data set with a second data set representing the patient's responses to stimuli from the stimuli generator at a second point in time and calculates a measurement of central nervous system variation in response to the comparison of the patients first and second data sets.

A still further aspect of the present invention includes a method for assessing cognitive change by processing data corresponding to a subject's brain activity evoked by a series of stimuli during first and second testing sessions. The method includes the step of identifying first subsets of data from the first testing session corresponding to times that a stimulus was presented to the subject. Similarly, second subsets of data from the second testing session corresponding to times that a stimulus was presented to the subject are identified. The first subsets are then compared with the second subsets. The comparison includes the step of using whole-brain assessments in a time, frequency or time-frequency domain. A measurement of difference between the first subsets and the second subsets is calculated. The measurement of difference is indicative of change in central nervous system function between the first testing session and the second testing session.

Another aspect of the present invention is an apparatus for assessing a change in central nervous system function. The apparatus includes means for providing a first set of stimuli to a patient to evoke a series of central nervous system responses. Means for monitoring brain activity provides data corresponding to the series of central nervous system responses. Means for processing processes the data from the means for monitoring brain activity to create a first data set representing the patient's responses to the first set of stimuli at a first point in time. The means for processing compares the first data set with a second data set representing the patient's responses to stimuli from the stimuli generator at a second point in time and calculates a measurement of central nervous system variation in response to the comparison of the patients first and second data sets. The comparison uses whole-brain assessments in time, frequency or time-frequency domains.

### DESCRIPTION OF THE DRAWINGS

The foregoing summary and the following detailed description of the preferred embodiments of the present invention will be best understood when read in conjunction with the appended drawings, in which:

FIG. 1 is a diagrammatic illustration of a system for assessing CNS condition;

FIG. 2 is a diagrammatic illustration of a map of event related potentials for a patient;

FIG. 3 is a diagrammatic view of a map of cortical connections for a patient;

FIG. 4 is a diagrammatic view of a map of cortical connection for a patient;

FIG. 5A is a plot of statistically significant differences between a baseline testing session and a subsequent session for a first subject;

FIG. 5B is a plot of statistically significant differences between a baseline testing session and a subsequent session for a second subject showing CNS changes associated with a concussion;

FIG. 6A is whole brain time frequency plot based on significance differences for a group of concussed subjects;

FIG. 6B is whole brain time frequency plot based on significance differences for a group of control subjects;

FIG. 7 is a graph of whole brain time frequency significance scores for concussed subjects (C1-C9) and control subjects (NC1-NC4);

FIGS. 8A-8I is a series of individual whole brain time frequency plots for nine concussed subjects;

FIGS. 9A-9D is a series of individual whole brain time frequency plots for four control subjects;

FIG. 10A is a graph of whole brain time frequency significance scores for concussed subject C3 having post-concussion syndrome showing changes over time;

FIG. 10B is a graph of whole brain time frequency significance scores for concussed subject C5 having post-concussion syndrome showing changes over time;

FIG. 11A is a graph of whole brain time frequency significance scores for concussed subject C1 showing changes over time;

FIG. 11B is a graph of whole brain time frequency significance scores for concussed subject C1 showing changes over time; and

FIG. 11C is a graph of whole brain time frequency significance scores for concussed subject C1 showing changes over time.

### DETAILED DESCRIPTION OF THE INVENTION

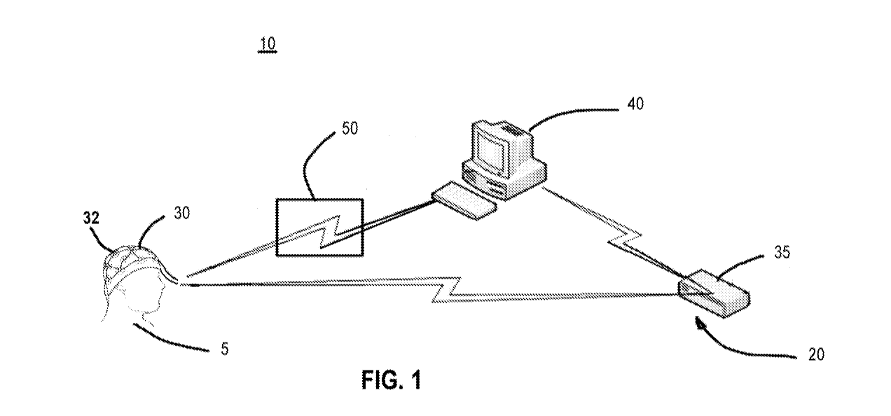

Referring now to the figures in general and to FIG. 1 specifically, a system for assessing central nervous system function (CNS) is designated 10. In the following discussion, it should be understood that the term central nervous system function or CNS also includes cognitive function, neurocognitive function and/or mental function. The system 10 is operable to aid in the diagnosis of a CNS condition or in the evaluation of the effectiveness of a treatment. For instance, the system may be used to aid in the diagnosis or monitoring of the progression of CNS deficit resulting from injury, such as a traumatic brain injury (TBI) or from disease, such as Alzheimer's disease. Similarly, the system 10 may be used to detect improvement after the TBI or monitor the effects of treatment designed to treat a neurodegenerative disease. The system provides an objective statistical metric of CNS function or symptoms by measuring the brain's activity associated with one or more tasks designed to elicit one or more specific CNS symptoms that are associated with the neurodegenerative condition or disease.

The system 10 includes a monitoring device 20 for monitoring the brain activity of a patient 5 in response to one or more stimuli provided to the patient. Data from the monitoring device 20 is analyzed to determine a measure of CNS condition for the patient. The patient data can be compared with similar data obtained for the patient at a different time to aid a medical professional in the diagnosis or treatment of a neurological or neurodegenerative condition.

Referring still to FIG. 1, the monitoring device 20 may be any of a variety of systems for monitoring the brain activity of a patient 5. For instance, the monitoring device may be an electroencephalogram (EEG) including event-related potentials (ERP) and evoked potentials (EP); a magnetoencephalogram (MEG) including event-related magnetic fields (ERF); any functional modality of a magnetic resonance imaging system (fMRI); a positron emission tomography system (PET); or an optical system for detecting near-infrared spectroscopy signals (NIRS) or event-related optical signals (EROS). In the present instance, the monitor 20 is an EEG.

The EEG 20 measures and records electrical field potentials of the brain. A variety of different types and sizes of electrodes can be used to detect the electrical field potentials. Although the system is operable with intracranial electrodes, such as in an electrocoticogram, in the present instance, the monitor 20 incorporates extracranial electrodes in the form of small electrically conductive discs or sensors. In particular, the system 10 incorporates a plurality of electrodes 32 configured to engage the patient's scalp. More specifically, in the present instance, a plurality of electrodes 32 are arranged in an array 30 configured to engage the scalp at a plurality of locations so that the electrodes are positioned to monitor electrical activity of the brain in various areas of the brain (e.g. neocortical areas such as occipital cortex including visual areas, parietal cortex including sensorimotor association areas, temporal cortex including auditory areas, and frontal cortex including motor centers). The electrode array 30 may have any of a variety of well-know electrode arrangements, and in the present instance, the array incorporates 16-24 electrodes configured according to the international 10-20 system.

Since scalp-recorded EEGs are greatly attenuated, principally due to the high resistivity of the skull and resistivity changes at the inner and outer boundaries of the skull, signals from the electrode array 30 are amplified by an amplifier 35. The amplifier 35 feeds the signals to a microprocessor, such as a personal computer 40 that processes the data from the EEG 20. Although the present system uses a personal computer, a variety of devices are operable to process the data from the EEG 20, including, but not limited to a tablet computer, an embedded processor, an online application using the internet or a cloud-based transmission, storage and/or analysis of the data. Accordingly, it should be understood that the processor for processing the EEG data may be any of a variety of electronic devices or systems configurable to process the EEG data.

The system 10 also includes an element for generating stimuli designed to evoke a CNS response from the patient. The system may provide any of a variety of sensory stimuli. In the present instance, the system provides visual stimuli in the form of visual cues displayed on an electronic display, such as the monitor of the personal computer 40. Further still, although the element for generating stimuli may be provided as a separate element, the personal computer 40 is programmed to provide a series of visual and/or auditory stimuli to evoke a series of neurological responses in the patient.

Referring to FIG. 1, the system 10 optionally includes a sensor 50 for detecting a physical characteristic of the patient other than brain activity. The sensor 50 may be a manually actuable element or it may be passively actuated by the patient. For instance, the sensor 50 may comprise one or more buttons or switches that the patient actuates in response to a stimulus. Similarly, the sensor may be a touch screen that the patient can touch to indicate a selection or response to a stimulus. In such an instance, the touch screen incorporates both the structure of the stimulus display and the structure of the sensor 50 for detecting a physical characteristic.

Additionally, or alternatively, the sensor 50 may comprise a passive element, such as a sensor for detecting the patient's eye movements, position or fixation while the patient is subjected to the series of stimuli. The sensor 50 may track the eye of the patient or it may track whether the patient's eyes are open or closed, or it may monitor both. The sensor may be any of a variety of sensors or detectors for monitoring eye movement, position or fixation. For instance, the system may be configured so that the eye detection mechanism is integrated into the display screen that the patient watches for the stimuli. Alternatively, the sensor may comprise one or more cameras that digitally monitor the patient's eyes.

In addition to or instead of the eye tracking sensor, the system may include a sensor for detecting a different physical characteristic of the patient. For instance, the system may include a sensor for detecting the position and/or orientation of the patient's head during the testing. In this way, the system can provide data as to whether the patient is engaged with the stimuli during the test or distracted.

The sensor is operatively connected with the processor 40 to provide data regarding the physical characteristic relative to the timing of the stimuli provided during a test. The data from the sensor may also be indicative of the substance of the patient's response (i.e. did the patient press a button when a response to a stimulus was appropriate). Based on the data from the sensor 50, the system may process the data from the brain function monitoring device 20 in combination with the data from the sensor 50 to assess the patient's cognitive functioning, as described further below. Additionally, the system may incorporate a device for detecting the timing of the stimuli and the system may be configured to monitor the timing that the patient actuates the sensor 50 so that the system tracks the timing of the patient response relative to the timing of the stimuli. The timing functionality may be incorporated in the functionality of the computer 40 monitoring the output from the brain function monitor 20 as a function of time. Alternatively, the timing functionality may be a separate mechanism such as a photocell that detects the presence of the stimuli and provides data to the system indicative of the time of the stimuli.

As described above, the sensor may be an active sensor that the patient or test subject manipulates or positively actuates, such as a button or other input device. Alternatively, the sensor may be passive, such as the eye tracking sensor that tracks the test subject's eye movement rather than requiring the test subject to actuate the sensor. Another passive sensor is a sensor that monitors the subject's balance or sway during the testing session. For example, a sensor may incorporate one or more accelerometers that detect movement by the subject. Specifically, for a test protocol that uses an EEG monitor, the EEG electrodes are commonly mounted on a cap that fits over the subjects head. The cap may include a sensor having two or more accelerometers that monitor movement in two or three orthogonal axes. Alternatively, the sensor may be mounted onto the patient separately from the cap. In this way, the sensor detects movement of the subject during the testing, which may be indicative of the patient swaying or having balance issues during the testing.

### Method of Operation

Configured as described above, a system 10 is provided that is operable to assess cognitive change in a patient. The system is used to perform a test on the patient to develop a baseline data set that provides a measure of the patient's CNS functioning. At a later date, the patient is tested a subsequent time according to a protocol similar to the first test and the data obtained during the subsequent test is compared with the data from the first test to evaluate the variation in CNS function of the individual.

To conduct a test, the patient is brought into operative engagement with the monitoring device 20. The particular steps for doing so will vary based on the type of monitoring device utilized. In the embodiment in which the monitoring device is an EEG, the array of electrodes 30 is applied to the scalp of the patient so that the contacts 32 are in electrical connection with the scalp. In the present instance, the electrodes 32 are attached to a fabric or mesh cap configured to be placed on the skull of the patient. Each electrode 32 comprises a separate electrical conductor that is connected with the EEG 20 so that the EEG can separately monitor the electrical activity detected by each electrode and provide data corresponding to such electrical activity to the computer 40.

During a test, the system provides a series of stimuli to the patient and the

EEG monitors the data regarding the electrical activity detected by each electrode. As noted above, based on data from the sensor 50, the system also measures the timing of a patient response and the accuracy of the response.

The electrical activity from CNS activity elicited from a task comprises event-related potentials (ERPs), in the time domain, event-related power spectral densities (ERPSDs) in the frequency domain, and event-related oscillations (EROs) in the time-frequency domain (also known as event-related spectral perturbations). For example, a sensory ERP called P100 occurs 100 ms after a particular visual stimulus (a reversing checkerboard pattern) is presented. By knowing the time between the checkerboard reversals and the recorded signal, the CNS processing time for the patient can be determined. The stimulation leading to an ERP can be either an external stimulation (such as a flash of light or an audible tone) or an internal stimulation (such as not providing a stimulus that the patient is expecting). In the case of providing an external stimulation, the alteration of the ongoing EEG in response to the external stimulation is also called an evoked potential (EP) in the time domain, and an evoked oscillation in the time-frequency domain.

To assess more complex brain function, more complex tasks are incorporated, which elicit cognitive ERPs. For instance, the “oddball” paradigm is a known series of tasks associated with attention and information processing capacity. During the oddball paradigm, the patient detects and responds to infrequent target events that are embedded in a series of repetitive non-target events. The stimuli are repeatedly presented in an imbalanced ratio, such as 80% to 20% and provide a metric of CNS resource allocation and decision making. Typically, larger ERPs are recorded for the rare target stimuli because the brain orients attention to what is less likely and task related (infrequent targets) in a preferential manner over that which is more likely and unrelated to the task (frequent non-targets).

The oddball paradigm entails top-down regulated attention to a stimulus.

Additionally, periodically, novel (individually unique) stimuli are presented that do not require a behavioral response (infrequent non-targets). By presenting novel deviants, the ongoing focus of the patient on the desired stimulus is broken and the deviant attracts the attention of the patient. Typically, the oddball paradigm elicits brain activity in a widespread cortical network. For instance, part of the task requires an inhibition process to suppress non-target deviant stimuli. If this type of task is applied to a schizophrenic patient, the inhibition may disappear so that little difference is seen between target and non-target stimuli.

Adding a secondary challenge, such as a memory task, to the oddball task, provides a method for assessing a more complex brain response that more directly relates to the symptoms to be evaluated for the patient. Furthermore, the combination of tasks can be designed so that the brain response with respect to decision making is evaluated, as well as the brain response with respect to memory. Similarly, additional tasks or components of tasks can be used to evaluate various responses, including, but not limited to attention, distractibility, vigilance, reasoning, emotional state and a host of other factors.

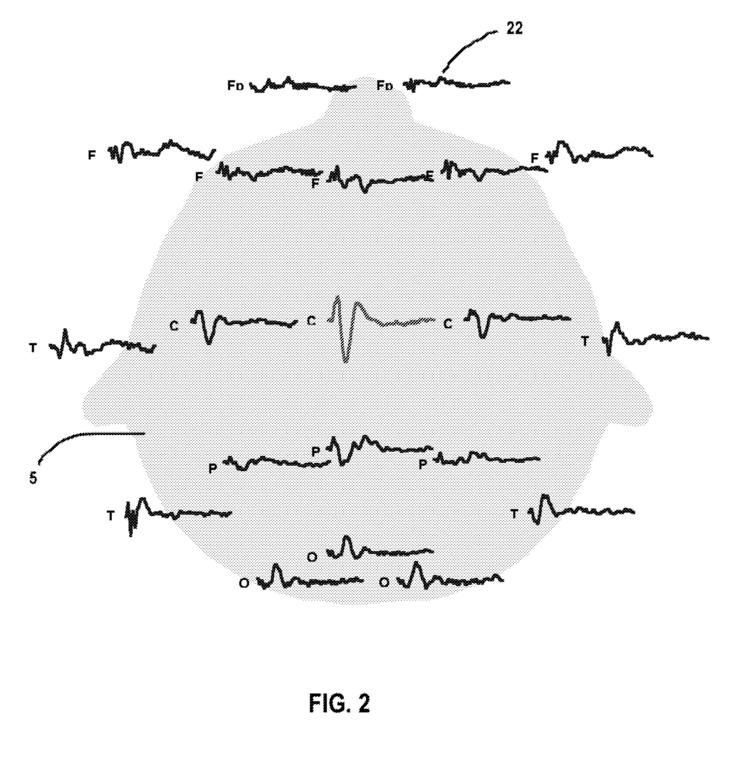

ERPs are typically quantified by amplitude and latency at each electrode recording. For instance, referring to FIGS. 1-2, the ERP for each electrode 32 is illustrated adjacent the corresponding electrode location. As can be seen in FIG. 2, the ERP recorded for the electrodes vary by location. In other words, the same stimuli cause different ERPs at different electrodes.

The ERPs are typically analyzed at each discrete electrode using only a subset of the activity recorded. More often only the amplitude and latency data are utilized. In contrast, the present system uses an inclusive metric of brain function that considers the global brain activity associated with a stimulus task across all of the electrodes and across the entire processing interval of the stimulus, which for example can be between 0 and 2000 msec. The global perspective of the ERP may include and/or collapse across multiple ERP components, including, but not limited to ERPs such as N100, P200, P300 and N400.

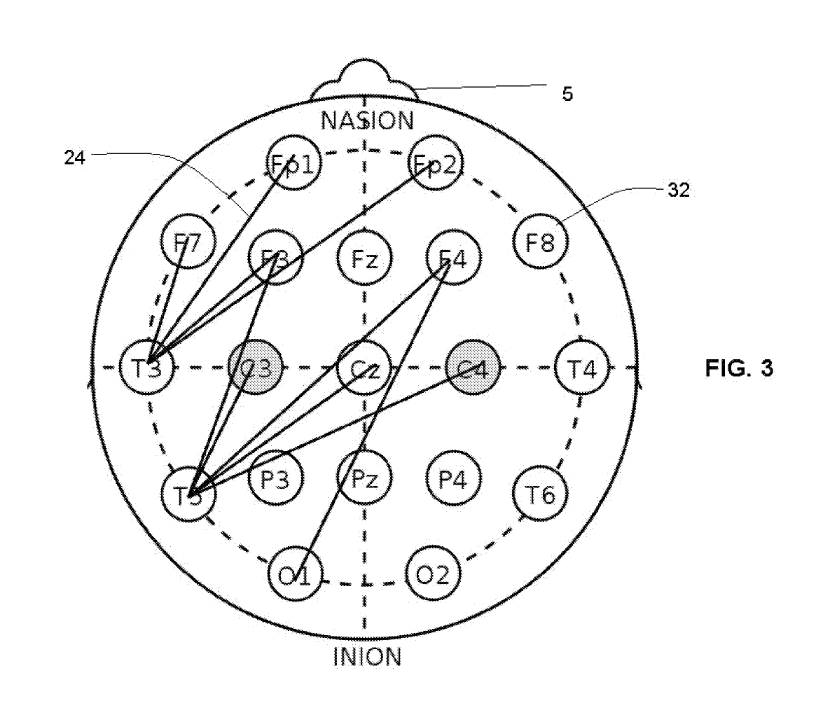

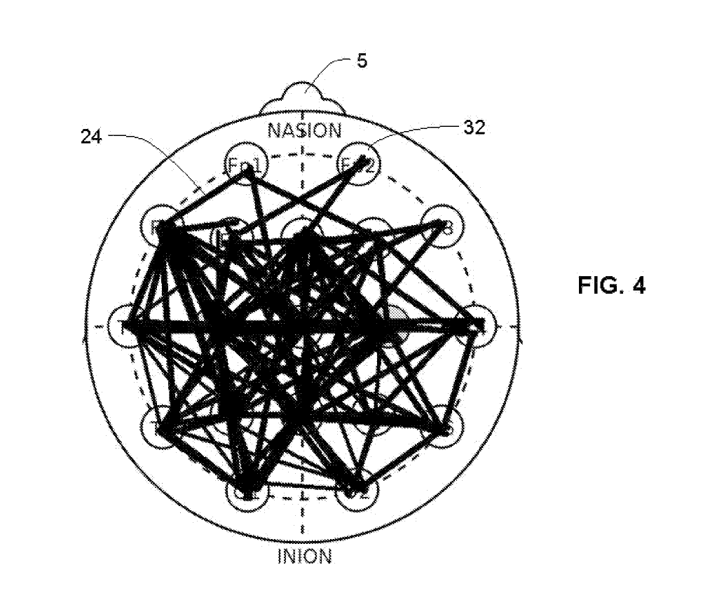

A more complete analysis of CNS function is provided by investigating how the areas of the brain are interoperating in response to the stimuli. The series of stimuli are designed to engage at least one or two primary cortical areas. However, these areas interoperate (i.e. connect with) other areas of the brain to accomplish the task. For example, referring to FIG. 3, the image illustrates a map of the cortical connections 24 between various areas of the brain. The cortical connection map may be derived from electrode data similar to the data illustrated in FIG. 2, which includes information about the relationship between regions of the brain, not just amplitude and latency. FIG. 3 illustrates baseline testing of functional connectivity, whereas FIG. 4 illustrates functional connectivity post-testing.

The connection between the various cortical areas can be described and/or measured in several different ways, including: anatomical connectivity, functional connectivity and effective connectivity. Anatomical connectivity refers to the network of physical connections linking different neurons or neuronal elements. Functional connectivity is a statistical measure of the interconnectivity between remote areas of the brain. If two areas are statistically independent, there is no functional connectivity between the two areas. Functional connectivity corresponds to the deviation from statistical independence between two areas. For instance, in the example of using an EEG, functional connectivity measures the interconnectivity of different areas of the brain based on the signal received by each electrode on the scalp. In other words, functional connectivity corresponds to the deviation from statistical independence between two areas based on the signals received from two electrodes. The statistical dependence can be estimated in a variety of ways, including measuring correlation or covariance, spectral coherence or phase locking. In contrast to anatomical and functional connectivity, effective connectivity is a measure of the influence one neuronal system exerts on another.

To provide a global perspective of CNS function, the data acquired by the EEG 20 is communicated with the computer and the computer processes the data to assess the functional connectivity between the brain areas involved over the course of processing the stimulus or task used to elicit the brain activity. Specifically, during the test the system provides the patient with a series of stimuli designed to evoke a series of CNS responses. The electrical activity for each electrode as a function of time is communicated with the computer. The data is combined with information regarding the stimuli and the timing of the stimuli to assess functional connectivity between the various sections of the brain. The post-stimulus EEG may be analyzed using the electrical activity recorded at the scalp or it may be based on source reconstruction methods that translate to a distribution of putative sources that are distributed on a mathematical representation of the cortical surface.

Depending on whether the scalp or a model is used, the computer processes the data to assess the cortical connectivity of areas involved. The system analyzes the data to assess the cortical connectivity of areas involved in the performed task(s). In the present instance, the measure of cortical connectivity is measured using whole-brain assessments in the time, frequency or time-frequency domains. Other methods may utilize a coherence analysis, synchrony, neural network models or other types of causality relationships.

After the system analyzes the cortical connectivity for the patient for the test, a metric can be calculated indicative of the CNS functioning of the patient. And when the patient is subjected to a second test in a manner substantially similar to the initial test, the data from the first test is compared against the data from the second test to assess the change, if any, between the CNS functioning of the patient between the time the first test was taken and the time of a subsequent test.

The data from the two tests can be analyzed in a variety of ways to evaluate the cognitive variation. For instance, the system may use non-parametric calculations or estimations based on data-driven probability calculations using randomization methods such as Monte Carlo, bootstrapping or permutation approaches to determine changes between the two sets of data for the patient resulting from the two tests.

One methodology for assessing the data to evaluate cortical and/or functional connectivity is referred to as whole-brain time-frequency change detection or Whole Brain TF for short. A Whole Brain TF methodology is designed to detect statistically significant changes of whole-brain functioning for a single subject over a plurality of testing sessions. For instance, an initial session may be used to provide a baseline set of data and subsequent testing sessions provide follow-up data that can be compared with the baseline to provide an assessment of changes in CNS relative to the baseline data to monitor potential improvement or degeneration of CNS function. Although a series of follow-up sessions may each be compared with the baseline data from the first session, it should be understood that the data for any two sessions can be compared with the first session considered as the baseline system and the subsequent session considered the follow-up data. In this way, it should be understood that the data for a series of sessions can be compared with the immediately proceeding session to assess the difference in CNS between the two sessions. Accordingly, it should be understood that in the following discussion the term baseline and follow-up are meant to connote a first session and a subsequent session. Additionally, the two sessions need not necessarily be separated by a time period. In some applications, it may be desirable to compare the data from a single testing session. In such an application, the first part of the testing may be considered the baseline session and the second part of the testing may be considered the follow-up session.

A statistical comparison of data from the follow-up session with data from the baseline session may provide a measurement of the similarity or difference of the two data sets. As discussed below, one such measure is a composite significance score that is based on a summation of significance scores derived from the baseline and follow-up data sets. Since the significance scores factor in data from all EEG channels simultaneously (i.e. data from each EEG electrode simulataneously), the composite significance score is a whole-brain measure.

The Whole Brain TF methodology comprises signal processing and statistical operations applied to a longitudinal series of single-subject task-related multichannel electroencephalographic (EEG) digitized recordings. Additionally, normalization procedures may be applied to the data. A statistical comparison of each follow-up session with respect to the baseline session is summarized as a composite significance score. The composite significance score is a summation of whole-brain atomic significance scores over the independent variables of time, frequency, and measure (for a range of times, a range of frequencies, and a pair of measures), in which
the time variable encompasses latencies on the order of milliseconds (ms) which are measured with respect to the onset of an event from a selected subset of events of a cognitive task performed by the subject;
the frequency variable reflects brain electrophysiological oscillations on the order of Hertz (Hz; cycles per second) which are observed in the temporal proximity of the selected subset of task events.
the measure variable encompasses two complementary ways of quantifying of event-related oscillatory activity: phase-locked activity and induced activity.
   Phase-locked oscillatory activity is derived from the average of single-trial event-related EEG epochs (i.e., the event-related potential (ERP)); and
   Induced oscillatory activity is derived from the standard deviation of single-trial event-related EEG epochs.

The Whole Brain TF methodology includes the following steps:

### Step 1: Identifying EEG Epochs.

The data from a session is analyzed to identify a series of EEG epochs that are event-related. An EEG epoch is a subset of the EEG data from a testing session (either baseline or follow-up). The epoch is the set of data time-locked to an event, such as a stimulus. The epochs may be identified manually or automatically. For instance, an operator may analyze the data to identify events of interest and the data corresponding to the events of interest may then be separated out as epochs of interest. However, in the present instance, the epochs are separated automatically as discussed further below.

The system collects EEG data as a series of electrical potential values measured by each electrode at discrete time periods. The data for a particular electrode is referred to as a channel and the electrical potential values for each channel are stored separately from one another. In this way, when N channels are used during testing, the stored data includes N subsets of electrical potential values.

When the data is analyzed, the analysis is performed on subsets of data of interest. In the present instance, the subsets of data are referred to as epochs, which are intervals of data aligned with selected stimuli of interest presented to the subjects.

As discussed previously, the system provides a series of stimuli to the subject during a testing session to evoke a series of responses. The system controls the timing of the presentation of the stimuli so the system is able to correlate the EEG data that is collected with the time a stimulus is presented. In particular, the system is configured so that stimulus events—with respect to which epochs are derived—are recorded with temporal precision in synchrony with the physiological data. Therefore each epoch includes a series of electrical potential values recorded over a period of time. More specifically, each epoch includes N sets of electrical potential values for a period of time for N channels used during the testing.

The time window for each epoch may include data recorded before a stimulus is presented as well as after the stimulus is presented. The system may be configured so the operator may adjust the time window relative to the presentation of the stimuli (for all epochs or on an epoch by epoch basis). However, in the present instance, the time window is automatically set so the time window is consistent for each epoch. In other words, each time window is the same length and begins at the same time relative to the presentation of each stimulus and ends at the same time relative to each stimulus. For example, each time window may be 500 msec in duration and may start 100 msec before the time a stimulus is presented. In this way, each epoch is a subset of data starting and ending at a preset time relative to the presentation of a stimulus, so that each epoch includes the same number of data points (i.e. the same number of discrete time/voltage data points).

Step 2: Time-frequency analysis of EEG epochs for a session to decompose the data in both time and frequency domains.

In the present instance, a wavelet transform is used for the time-frequency analysis to decompose the data in both time and frequency domains. However, it should be understood that any of a variety of transformations may be utilized for the time-frequency analysis, including, but not limited to Fourier transform (including the short time Fourier transform), Hilbert transform and complex demodulation. The wavelet transform of the present method is a continuous wavelet transform with Morlet wavelets that comprise sinusoids of different frequencies multiplied by Gaussian windows of different durations.

The processor transforms the data from an epoch on a channel by channel basis. The following discussion describes how the data in a channel of an epoch is processed. The time-frequency analysis transforms the time/voltage data from the EEG into a series of time/frequency coefficients using the Morlet wavelet transformation. In particular, each complex Morlet wavelet corresponds to a particular combination of time and frequency which is referred to as a support point on the time-frequency plane. For each support point, the transform applies the corresponding Morlet waveform to the epoch of data to derive a corresponding complex wavelet coefficient. In this way, the transformation results in a series of complex wavelet coefficients across all channels for each support point of each epoch.

The phase-locked measure at each support point and channel across all of the epochs is obtained by averaging the real and imaginary parts of the derived complex wavelet coefficients. The induced measure is obtained by calculating the standard deviations of the real and imaginary parts.

By way of example, a testing session may include 50 epochs, with each epoch including 128 time/voltage data points for each channel and the test may include 24 channels. The wavelet transformation may employ 64 time-frequency support points for each channel of each epoch, in order to derive 64 wavelet coefficients which are complex numbers in the form a+bi. To calculate the phase-locked measure at each support point, the wavelet coefficients are averaged over all of the epochs on a channel by channel basis. Therefore, all wavelet coefficients for the first support point of channel 1 for all 50 epochs are summed and then divided by 50 to obtain the mean of the wavelet coefficients. This is repeated for each support point for all channels so that the result is a series of 64 sets of 24 mean wavelet coefficients of the form a+bi that represents the phase-locked measure for the testing session. Similarly, the induced measure is calculated across all of the epochs by calculating the standard deviations of the real and imaginary components of the wavelet coefficients on a channel by channel basis for each support point. The result is a series of 64 sets of 24 standard deviation wavelet coefficients of the form a+bi that represent the induced measure for the testing session.

### Step 3: Difference Magnitude Analysis

The data for a baseline session is compared with the data from a follow-up session after the time-frequency analysis discussed above in Step 2 is performed for the data for each session. The data from the two sessions are then compared to calculate a difference measure between the two data sets. The difference is calculated across all channels so that the measure reflects whole-brain changes. In this way, the difference reflects the underlying changes in cortical activity and/or connectivity.

Several methodologies can be employed to calculate the difference measure between the two data sets. For instance, the magnitude of the difference between the complex channel vectors for the baseline and follow-up sessions can be calculated. Alternatively, the magnitude of the difference can be calculated by utilizing the entire complex covariance matrix as discussed further below.

In the present instance, the difference measure is determined by calculating the magnitude of difference between the complex channel vectors. More specifically, at each of the 64 support points (for either the phase-locked measure or the induced measure) the 24 channels result in a 24-dimensional channel vector. For each support point, the 24-dimensional channel vector of the baseline test is compared with the 24-dimensional channel vector of the follow-up test to determine the difference magnitude between the two vectors. This is done separately for the phase-locked and induced measures. Specifically, the difference magnitude between the baseline and follow-up sessions is the square root of the sum of the moduli squared of complex differences per each channel. For example, in the instance described above in which there are 24 channels and 64 support points for each channel for the phase-locked measure and the induced measure, the difference magnitude for the phase-locked measure is:

$$Difference Magnitude = \sqrt{\sum {\{ {a}_{jbaseline} - {a}_{jfollow - up} )}^{2} - {( {b}_{jbaseline} - {b}_{jfollow - up} )}^{2} \}}$$

where for the phase-locked measure
a_{j baseline} is the real component of the mean wavelet coefficient from the j^{th} channel of the baseline data;
a_{j follow-up} is the real component of the mean wavelet coefficient from the j^{th} channel of the follow-up data.
b_{j baseline} is the imaginary component of the mean from the j^{th} channel of the baseline data; and
b_{follow-up} is the imaginary component of the meanwavelet coefficient from the j^{th} channel of the follow-up data.
For the induced measure aⱼ and bⱼ represent the respective standard deviation wavelet coefficients.

The result is 64 difference magnitudes, each one corresponding to a time-frequency support point for the phase-locked measure and also for the induced measure. These difference magnitudes are nonnegative numbers that reflect the whole-brain difference between two sessions (follow-up versus baseline) for a given measure of event-related oscillatory activity (phase-locked or induced). Since the vector difference magnitude is based on whole-brain changes, the differences identified reflect underlying changes in cortical activity and/or connectivity.

### Step 4: Calculating Significance Scores

A whole brain significance score is based on the estimated probability (p-value) that an observed difference magnitude between baseline and follow-up data for a particular time-frequency point resulted from chance. In the present instance: significance score=−log (p-value).

When the probability that an observed difference resulted from chance decreases the statistical significance of the difference increases. For example, when the p-value decreases from 0.1 to 0.01 to 0.001, the significance score increases from 1 to 2 to 3. In other words, for a difference magnitude between a baseline session and the corresponding follow-up session, as the p-value decreases, the likelihood that the change observed between the baseline and follow-up sessions resulted from a change in CNS function increases. Therefore, the significance score for the difference magnitude of an epoch increases. A composite significance score is calculated by summing the significance scores over a subset of time-frequency support points for a range of times and a range of frequencies for both phase-locked and induced measures.

Calculating p-Values Used to Create Significance Scores

A variety of methodologies can be used to estimate the p-value for a difference magnitude that is calculated as discussed above. In the present instance, nonparametric random permutation testing is used to estimate the p-values. Such testing does not make assumptions about how the difference magnitudes are distributed.

The first step of the permutation testing is to generate a nonsignificant difference magnitude. A nonsignificant difference magnitude is defined as a difference magnitude that occurred due to chance (as opposed to a change in CNS function between the baseline and follow-up sessions). The nonsignificant difference magnitude is generated by:
(a) randomly permuting the indices of all single-trial epochs across both sessions so that “baseline” and “follow-up” assignments are reassigned; and
(b) reprocessing the data to obtain a difference magnitude (as if the random reassignment of sessions had been the actual assignment).

In other words, in a first permutation, the indices from all of the epochs for the baseline and follow-up data are randomly divided into two groups and the two groups are then compared are discussed above in step 3. The indices refer to the support points which underlie the complex wavelet coefficients determined in step 2. These wavelet coefficients do not need to be re-calculated for each permutation.

In the present instance, this randomization procedure is repeated N times (e.g., N=199). By permuting the data N times, the process generates an empirical distribution of difference magnitudes that would be observed when the null hypothesis is true. In other words, an empirical distribution is generated of difference magnitudes that would be observed when there is no change from baseline to follow-up sessions. Such a distribution is referred to as the null distribution.

A p-value estimating the probability that an observed difference magnitude is due to chance is estimated as p=C/(N+1) where C counts the number of times a nonsignificant difference magnitude equals or exceeds the actually observed difference magnitude. For example, if N=199 and C=9, then p=9/200=0.045, which has a significance score of −log(0.045)≈1.347.

If this procedure is repeated for all time-frequency support points independently, the obtained p-values are uncorrected for multiple comparisons. Correction of p-values for multiple comparisons is accomplished by generating a single distribution of the maximum nonsignificant difference across all time-frequency support points for each randomization.

Specifically, returning to the example in which each epoch has 64 support points for the phase-locked measure, step 4 will result in 64 difference measures, which in turn result in 64 p-values. In order to obtain p-values which are corrected for 64 simultaneous comparisons, one common distribution of null hypothesis difference magnitudes is obtained for all 64 support points. For example, if 199 randomizations are performed, the result is 199 sets of 64 difference magnitudes for the phase-locked measure. For each randomization, the maximum of the 64 difference magnitudes is retained to produce the empirical null distribution comprising 199 maximum difference magnitudes.

The 64 p-values are converted to 64 significance scores by the relation significance score=−log(p-value). To calculate a composite significance score, a subset of the significance scores are selected. The selected significance scores correlate to support points that fall between a specified range of times and a specified range of frequencies. The select significance scores are then summed to form the composite significance score. The subset of significance scores that are summed to form the composite significance score may include all of the significance scores. However, in general, the selected significance score are a subset. So the composite significance score is the sum of (“atomic”) significance scores over all support points within the specified time-frequency rectangle for the phase-locked measure plus the same for the induced measure.

As described above, the system may include a detector for monitoring a physical characteristic separate from brain activity measurements. Data from this detector may be used in combination with the analysis of the brain activity data described above. For instance, if the detector or sensor 50 is a button, the system may utilize data from the sensor in combination with the data regarding the brain activity to assess the cognitive function of the patient. For instance, the system may categorize the brain function data in one way if the button indicates a correct response whereas the data may be categorized differently if the button indicates an incorrect response. Additionally, the timing of the patient response may be incorporated into the analysis of the data when assessing the cognitive functioning of the patient.

Similarly, if the detector 50 is an eye tracking device or a sway detector, the data regarding the eye movement or sway can be used to evaluate the validity of the assessment. More particularly, if the data from the eye tracking device indicates that the patient's eyes are not properly focused on visual stimuli when the stimuli was presented, the system may invalidate and/or discard the data corresponding to the task in which the patient's eyes were not focused on the visual stimuli. Similarly, or alternatively, the data from the eye tracking device can be used to validate data if the data from the eye tracking device shows that the eyes were properly focused on the visual stimuli when the stimuli was presented to the patient.

### EXAMPLE

One candidate for an objective measurement of CNS status is the assessment of concussion using event related potentials computed from electroencephalography recordings.

In the known systems using EEG methods, a number of assumptions were made regarding spontaneous brain states (i.e., that they are consistent and predictable within the recording) and about the relation of an individual to normative data (i.e., that every individual falls within the norm) when using QEEG analysis. In contrast, the present analysis is individualized based on the creation of an empirical distribution of difference magnitudes that would be observed when the null hypothesis is true. As described above, the empirical distribution is based upon the data collected for the test subject rather than the results of other individuals. Additionally, in contrast to spontaneous brain states utilized in the known systems, the present system uses specific stimuli to elicit an event related potential.

Although the system may be used to evaluate the likelihood that an individual has suffered an injury or degenerative condition that has caused alterations in normal brain function, such as a concussion, the methodology is not a direct assessment of concussion itself, but rather changes in CNS function as a consequence of a concussive injury. By evaluating ERP's, the testing can be designed so that specific brain states are evoked by different stimulus presentation, allowing specific cognitive functions to be evaluated simply by changing the stimuli presented, or a set of CNS functions can be evaluated by combining various stimuli.

Additionally the system can be used to evaluate the beneficial effects of medications or interventions on the function of the CNS, by assessing positive changes from an altered brain state associated with treatments.

ERPs are stable measures so they can allow an individualized assessment tool when combined with serial testing. ERPs therefor are good candidates for serial assessments of individuals, comparing a post event assessment to a prior recording or baseline. Thus, ERPs provide both a method for assessing both self-referenced data, as well as task-specific data, which can identify CNS changes by directly recording the brains electrical activity.

The present example summarizes the results of an investigation that provided a comparison that included a comparison of self-referenced and task specific data to post-concussion data in a prospective manner.

### Subjects

The test subjects were student athletes were recruited from a university football program. Subjects were between the ages of 18-24 years and each received a baseline testing prior to the first practice in which contact was allowed. This provided a window of about six months since the end of the previous football season during which the athletes were not practicing football. Therefore, the subjects had a low probability of undocumented concussion history. Each athlete answered a concussion history questionnaire that addressed both recent and past history of concussions. Injured subjects were identified by a certified athletic trainer or medical staff, according to the standard concussion protocols currently in place. For purposes of the test, concussion was defined as an injury resulting from a blow to the head causing an alteration in mental status and 1 or more of the following symptoms prescribed by the American Academy of Neurology Guideline for Management of Sports Concussion: headache, nausea, vomiting, dizziness/balance problems, fatigue, trouble sleeping, drowsiness, sensitivity to light or noise, blurred vision, difficulty remembering, or difficulty concentrating. Criteria contributing to the identification of a player with a concussion also included the observed mechanism of injury (e.g., acceleration or rotational forces applied to the head), symptoms reported or signs exhibited by the player, and reports by medical staff or other witnesses regarding the condition of the injured player. Loss of consciousness (LOC), posttraumatic amnesia (PTA) (e.g., inability to recall exiting the field, aspects of the examination), and retrograde amnesia (RGA) (e.g., inability to recall aspects of the play or events before injury, score of the game) were documented immediately after injury.

Injured subjects that were identified during the study had a medical evaluation by the team physician and were treated according to current published guidelines, as they had been treated prior to onset of the study. Management of the players post-concussion was not influenced by results of the study.

To evaluate recovery in the post-injury interval, athletes identified with a concussion were tested up to a total of 3 times; (i) 24-48 hours post event, (ii) 5-7 days post event, (iii) 10-12 days post event. Subjects showing prolonged concussive symptoms had additional follow up testing over the course of their recovery determined on an individual basis. Some subjects that were allowed to return to play under the concussion existing protocol did not receive all three protocol assessments. To provide a source of control subjects, several non-concussed athletes will also be tested at least one time in addition to the baseline.

### Data Collection:

During the testing, EEG and ERP data was obtained by placing 24 conductive electrodes on the scalp using a conductive water-soluble electrolyte at locations based on the International 10/20 recording system. The electrodes were contained in an electrode array produced by Compumedics and the QuikCell electrode application system was used. Impedances were below 50 kOhms, with a target impedance of 20 kOhms. Continuously recorded EEG was sampled at 2000 Hz per channel simultaneously from all 24 electrodes. Additionally triggers from the stimulus onset (via photo diode) and a response device were collected as well.

### Test Protocol

An initial validation protocol was used to help determine data quality prior to the stimulation protocol. The validation protocol asked the subject to blink each time the subject saw the word blink on the screen. The word blink appeared three times for duration of 250 ms and an isi of 1000. This presentation was repeated three times with a 5000 ms rest between each block.

A second validation protocol was an eyes open, eyes closed protocol. Instruction was provided to the subjects to close their eyes and press a button to continue. A stimulus trigger is then sent out initiating 45 seconds of Eyes Closed. This was repeated with Eyes Open.

EEG data was collected across 24 channels while test subjects were presented with a variety of stimuli during each testing session. The testing stimuli included an odd ball task using upright (target) and inverted triangles (distractor) where the targets were to be responded to with a button press and the distractors were to be ignored. Additionally, novel non-targets were presented, including a non-triangular shape that was not repeated during the study. Typically, oddball protocols use an 80/20 percent ratio between the distractor and target. However, the introduction of the novel none target, changed the ratios to 68/16/16 for the distractor, target and novel non-target stimuli respectively. Because memory and multi-processing tasks were of interest in the assessment of CNS function, the standard oddball task was augmented with a secondary memory task, that included letter pairs (TH, MG OW etc.) shown inside 10% of the target and distractor triangles. Subjects were asked to remember the letter pairs during the oddball task and then were asked to recognize the letter pairs when a recall list was presented after each block of the oddball.

The test subjects responded to the target stimulus by pressing a button, whereas no response was necessary for the distractor. After the odd-ball task was completed a recall list was presented and subjects were asked to respond with a button press when they saw letter pairs presented in the original odd-ball task.

The odd-ball stimuli were delivered in 8 blocks of approximate 1 minute each. At the conclusion of each oddball task the letter pair recall list was be presented and lasted approximately 30 seconds.

### Data Analysis

An analysis similar to the steps described above was performed on the recorded data across, time, frequency and spatial domains. The Whole-Brain Time-Frequency (WBTF) analysis processed the data in a comprehensive manner and compared over two points in time for the same individual. The WBTF analysis used ERP data, averaging repeated blocks of data associated with a specific task and extended the typical ERP analysis to include the full complement of time and frequency data contained within each trial and across all channels, simultaneously.

The WBTF analysis used permutation testing of an individual's data to establish an individual distribution that was then compared to subsequent testing, such as post-event testing. The comparison incorporated the steps described above to compare baseline testing data (before an event) with post-event testing data (i.e., an event in which concussion symptoms were identified).

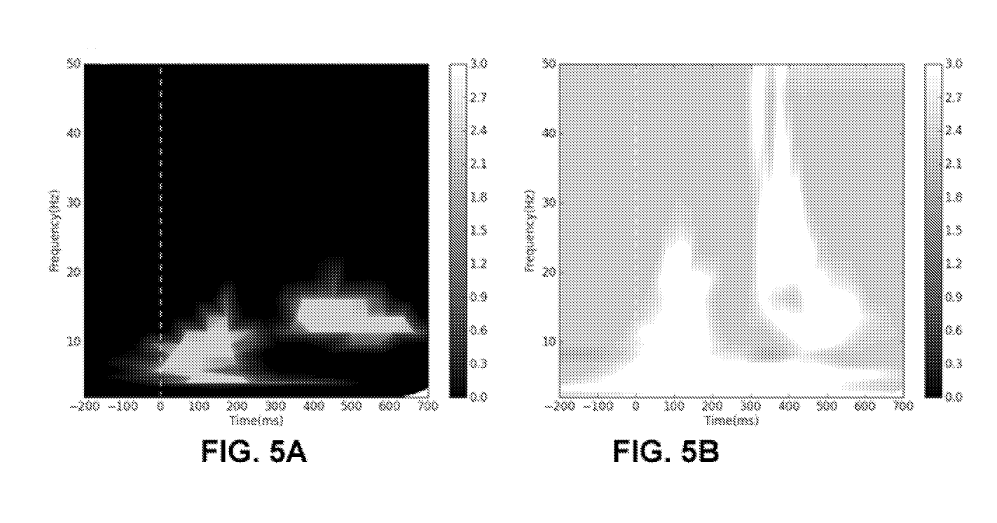

Two examples of the WBTF analysis output are shown in FIG. 5, where statistically significant changes between a baseline and post-event testing session are plotted for the differences in CNS processing associated with the stimulus task between the two time points. Less significant values are plotted in darker shades while areas of the greatest significance are plotted in lighter shades as shown by the key next to each plot. Due to normal variability in test/re-test reliability some statistically significant change is expected in all comparisons, however, a preponderance of significant changes may be associated with the concussion as seen in FIG. 5. In addition to the graphical depiction of the changes, these plots can be quantified but summing the significance scores to a single number. The number indicates the total value of significant change between the baseline and post-event testing.

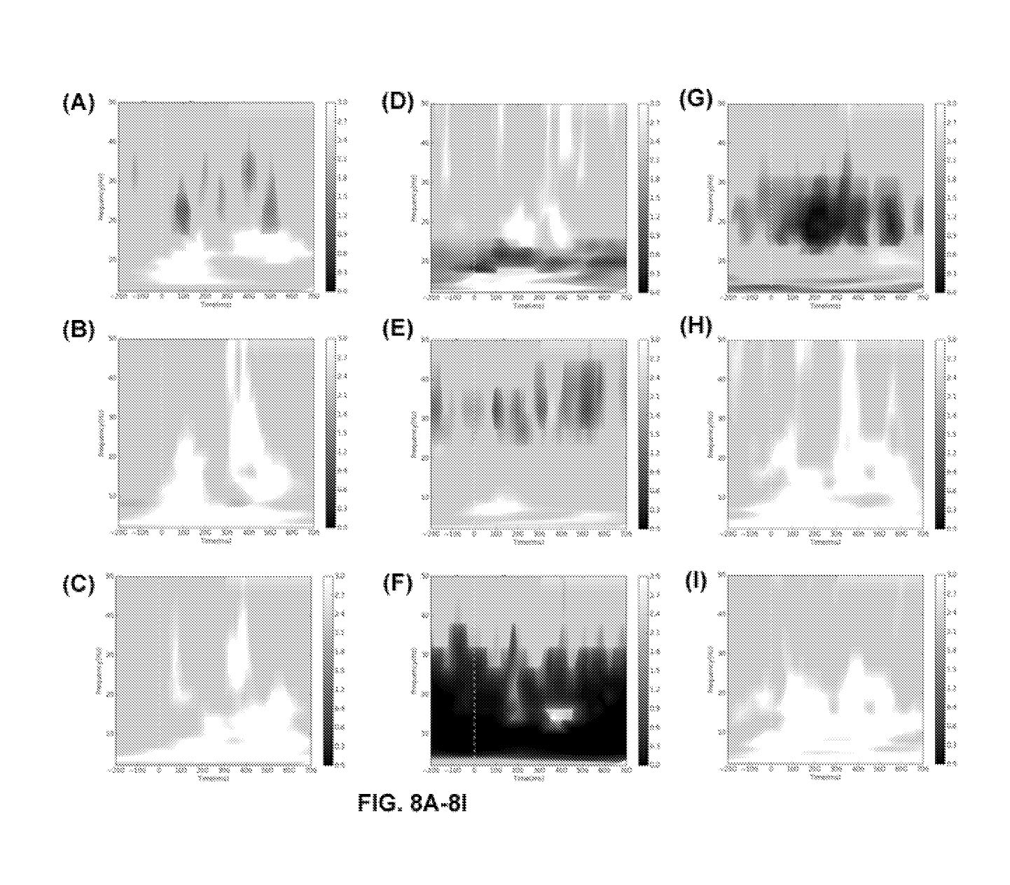

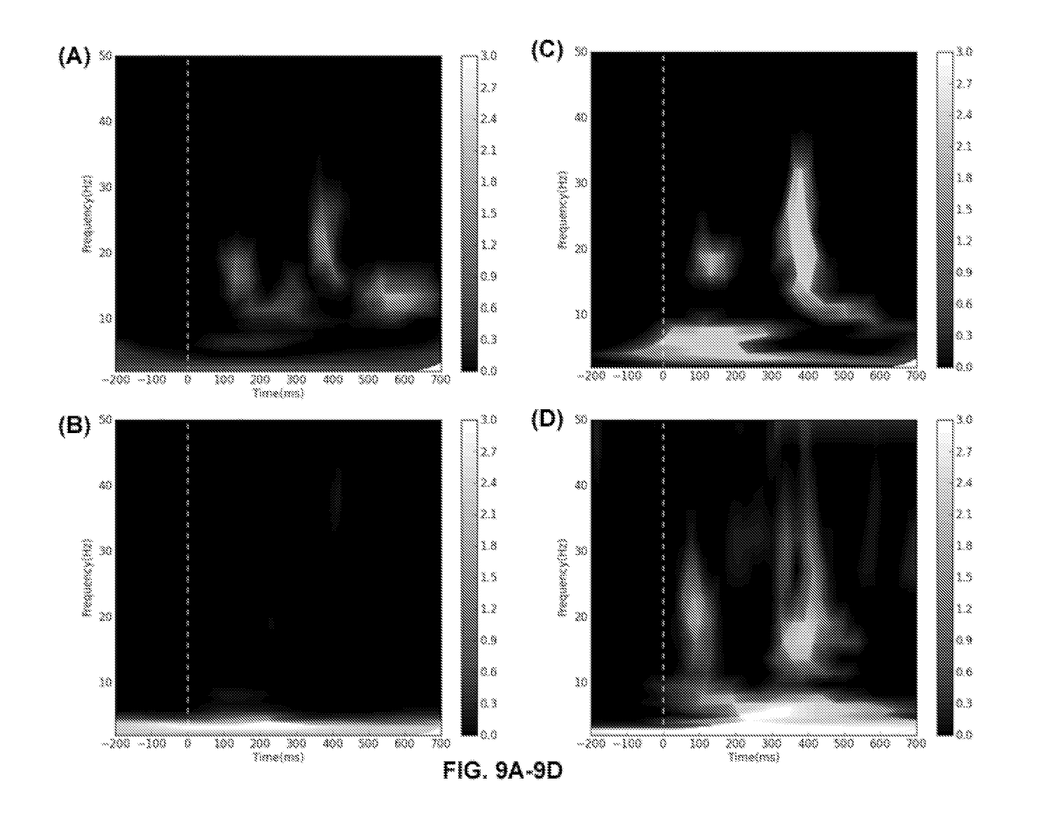

FIGS. 8A-8I show the WBTF plot for each individual that was identified as concussed and provides an opportunity to visualize the individual differences in the assessment of CNS change. While WBFT plots of the concussed subjects all show high significance values, the significance in the time-frequency domain is distributed. The largest changes in these plots are often associated with the classic sensory (i.e., 100 msec) and cognitive (i.e., 300 msec) time ranges for ERPs. FIGS. 9A-D show the individual WBTF plots for the control subjects.

### Prolonged Concussion Assessments

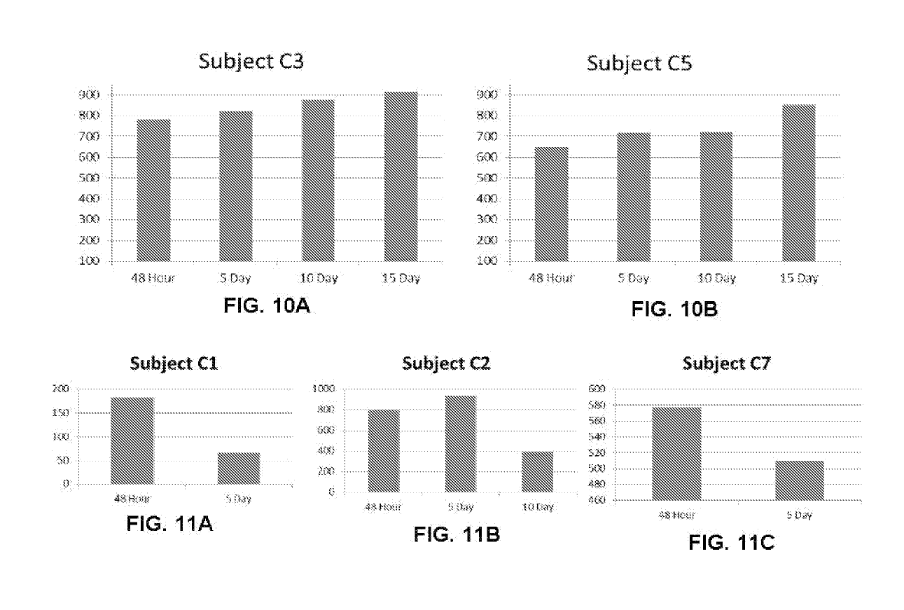

Five of the concussed subjects underwent additional testing sessions beyond the 48 hour time period. Two distinct patterns are evident in these follow up tests. First, two subject (C3, C5) were assessed four times post-concussion covering a time frame of 15 days post-injury. The assessments in these subjects show an increased change from the 48 hr assessment as shown in FIGS. 10A-B. Neither of these athletes was cleared to play using the normal protocols in place for an extended duration and in fact subject C3 was medically disqualified from play for the season.

In contrast to the subjects presented above the remaining three subjects show a very different pattern, one that suggests recovery. Each of these subjects assessments are shown in FIGS. 11A-C and indicate a reduction in the significant change from the baseline testing to the last assessment period. These subjects were returned to play after the last assessment period, so no further testing sessions were performed.

### Discussion

The use of an objective brain-based measure was shown to be a useful in the assessment of CNS function and in particular to identifying changes in CNS due to concussion. Previous methodologies based on subjective report or behavioral performance on cognitive tasks have been shown to be less than effective. Further, these measures are often shown to resolve or return to a baseline state quickly, often within 7 to 10 days. However, while the subjective effects or behavioral performance may resolve, the physiological effects may linger much longer. This potential long term physiological consequence may contribute to the long term effects of concussions.

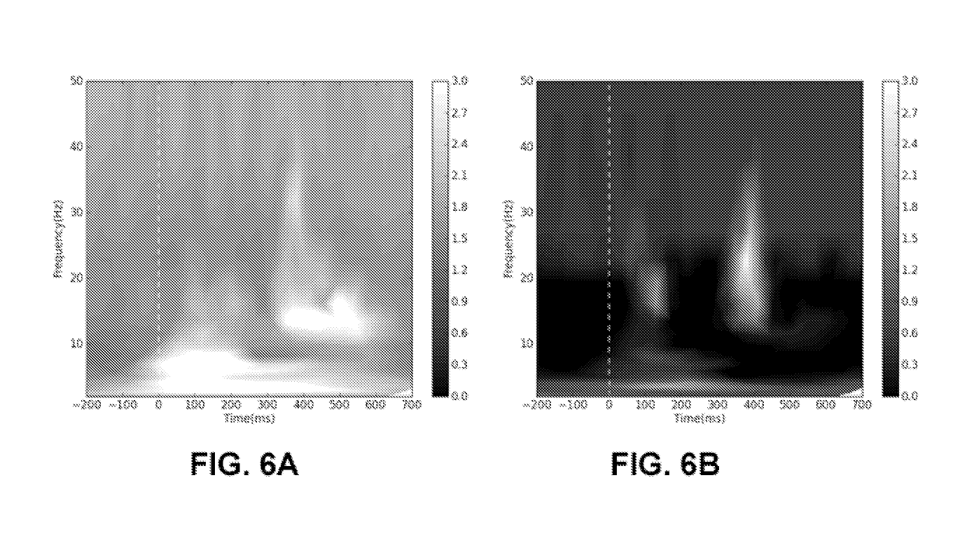

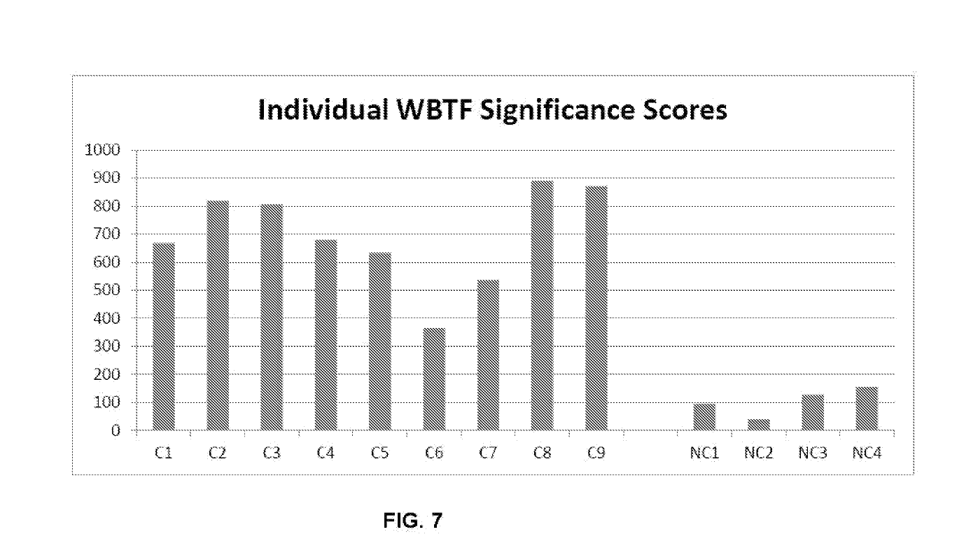

The present system 10 improved sensitivity to CNS change by evaluating an individual's electrical brain activity associated with specific task challenges. The changes are evaluated on an individualized basis rather than being evaluated relative to normative data from a group of subjects. The system's operation engages multiple brain functions of the patient, such as memory, recognition, executive function, inhibition and sensory processing. As shown in FIGS. 6A-9C, based on initial post-concussion testing within 48 hours of injury, the present system was 100% sensitive in identifying CNS changes in individuals with physician diagnosed concussions. It was also shown that the averaged individual changes in the concussed group was significantly different that the averaged individual change in the control group, even with a major outlier in the control group. WBTF significance scores for the concussed subjects showed expected variability ranging from over 350 to nearly 900, accounting for individual differences associated with the individual injuries and their effects. These summed significance values provide a comprehensive measure of the changes in CNS function associated with the distractor task across all recording sites in the time-frequency domain.

As described above, the system provides an individualized assessment of significant CNS changes associated with concussion. However, averaging these individual results together provides a reference point for comparison. While the control group shows some changes between their baseline and second assessment, the magnitude of these changes are significantly less then the magnitude of changes in the concussed group.

The difference between the concussed and control group areadily observable by comparing the WBTF plots in FIGS. 8 and 9. These visual differences are helpful in understanding the results of the analysis. Although the WBTF significance score provides an absolute value, there is significant data underlying the singular score, and the plot represents this data.

Although only a subset of the concussed subjects had multiple assessments, two patterns emerged. One group showed increasing changes from baseline up to 15 days post-concussion, even though self-reported symptoms may have resolved. This appears to indicate that CNS recovery extends beyond the resolution of self-reported symptoms. This is particularly concerning given the recent findings that Chronic Traumatic Encephalopathy (CTE) has been linked to multiple concussive and sub-concussive injuries. Failure to wait until full physiological recovery occurs from a concussion may lead to increased effects of a second concussion (i.e., Second Impact Syndrome) and may eventually relate to the development of CTE. While the resolution of symptoms has largely been used to determine return to play guidelines, this may not allow an appropriate time for physiological recovery. A more sensitive measure based on objective physiological assessment, such as the system described above provides an important assessment tool for understanding the physiological recovery from both a single concussive event and the possible effect of cumulative events.

The second set of subjects showed a reduction in the changes from baseline, indicating a recovery of the CNS functions being assessed. Although these subjects were not followed up for an extended period of time, this trend may continue until the changes between baseline and post-concussion testing is greatly reduced or showed no differences. These patterns of recovery show that the system may also be used in the absence of a pre-injury baseline. If the initial assessment (i.e., baseline) is post-injury subsequent assessments show a positive change (i.e., increasing significance score) and this change can be tracked until it plateaus indicating CNS changes has ceased potentially indicating recovery is complete.

It will be recognized by those skilled in the art that changes or modifications may be made to the above-described embodiments without departing from the broad inventive concepts of the invention. For instance, in the methodology described above, difference magnitudes are calculated by determining the magnitude difference between complex channel vectors. Alternatively, the difference magnitude can be calculated for the induced measure by using the entire complex variance-covariance matrix. A matrix difference magnitude is computed as the Frobenius matrix norm of the difference matrix. Inclusion of the off-diagonal complex covariances incorporates measures of functional connectivity. It should therefore be understood that this invention is not limited to the particular embodiments described herein, but is intended to include all changes and modifications that are within the scope and spirit of the invention as set forth in the claims.

1-18. (canceled)

19. An apparatus for assessing a change in central nervous system function, comprising:
a stimuli generator operable to provide a first set of stimuli to a patient to evoke a series of central nervous system responses;
a brain function monitor operable to monitor the patient's brain activity and provide data corresponding to the series of central nervous system responses;
a processor configured to process the data from the brain function monitor to create a first data set representing the patient's responses to the first set of stimuli at a first point in time, wherein processing the data comprises assessing a cortical connectivity measure;
wherein the processor is configured to compare the first data set with a second data set representing the patient's responses to stimuli from the stimuli generator at a second point in time and calculate a measurement of central nervous system variation in response to the comparison of the patients first and second data sets, wherein the measurement of central nervous system variation is based on an assessment of a cortical connectivity measure based on the data from the brain function monitor.

20. The apparatus of claim 19 wherein the stimuli generator is a display for displaying visual stimuli.

21. The apparatus of claim 19 wherein the processor is configured to identify subsets of the first and second data sets corresponding to times that a stimulus is presented to the patient, perform a time-frequency analysis on the subsets of the first and second data sets to transform the data in a time-frequency domain, and calculate an observed difference measurement by comparing the transformed subsets of the first data set with the subsets of the second data set to assess the differences between the subsets of the first data set and the subsets of the second data set.

22. The apparatus of claim 21 wherein the processor repeatedly permutes the transformed subsets of the first and second data sets, wherein during each permutation the transformed subsets are randomly assigned into first and second groups of subsets and the processor compares the first group of subsets with the second group of subsets to assess the differences between the subsets of the first and second groups to generate a distribution of differences based on the comparisons of the permuted subsets of data.

23. The apparatus of claim 22 wherein the processor generates the measurement of central nervous system variation by comparing the differences assessed from the comparison of the transformed subsets of the first and second data sets with the distribution of differences.

24. The apparatus of claim 23 wherein the processor calculates the measurement of variation by generating a measurement of difference between the first set of data and the second set of data by comparing the observed differences with the distribution of differences.

25. The apparatus of claim 19 wherein the processor performs the data comparison by performing a whole-brain time-frequency analysis of data from the first and second sets of data.

26. The apparatus of claim 19 wherein the brain function monitor comprises an electrode array configured to engage the patient's scalp.

27. The apparatus of claim 19 comprising a sensor for detecting a behavioral characteristic of the patient while the brain function monitor monitors the patient's brain activity and the processor is operable to calculate a measurement of central nervous system variation in response to the comparison of the patients first and second data sets in combination with a comparison of data from the sensor.

28. The apparatus of claim 27 wherein the sensor is configured to examine eye movement of the patient and provide data relating to the eye movement of the patient.

29-37. (canceled)

38. A method for assessing cognitive change by processing data corresponding to a subject's brain activity evoked by a series of stimuli during first and second testing sessions, comprising the steps of:
identifying first subsets of data from the first testing session corresponding to times that a stimulus was presented to the subject;
identifying second subsets of data from the second testing session corresponding to times that a stimulus was presented to the subject;
comparing the first subsets with the second subsets, wherein the step of comparing comprises the step of using whole-brain assessments in a time, frequency or time-frequency domain; and
calculating a measurement of difference between the first subsets and the second subsets, wherein the measurement of difference is indicative of change in central nervous system function between the first testing session and the second testing session.

39. The method of claim 38 wherein the step of identifying the first subsets comprises selecting data relating to a select time window during which time window a stimulus was presented to the subject.

40. The method of claim 38 wherein the step of comparing the subsets comprises the steps of:
performing a time-frequency analysis on the subsets of data from the first and second testing sessions to transform the data in a time-frequency domain; and
calculating observed differences by comparing the first transformed subsets with the second transformed subsets to assess the differences between the first and second.

41. The method of claim 40 wherein the step of comparing the transformed subsets comprises the steps of:
randomly permuting the transformed subsets of the first and second data sets so that the subsets of the first and second data sets are randomly assigned into a first group of subsets and a second group of subsets; and
comparing the first group of subsets with the second group of subsets to assess the differences between the subsets of the first and second groups.
repeating the steps of randomly permuting the transformed subsets and comparing the first group of subsets and the second group of subsets, wherein the step are repeated a plurality of times; and
generating a distribution of differences based on the differences assessed during the steps of comparing the first group of subsets with the second group of subsets.

42. The method of claim 40 wherein the step of calculating a measurement of difference between the first subsets and the second subsets comprises the step of generating a measurement of difference between the first set of data and the second set of data by comparing the observed differences with the distribution of differences.

43. The method of claim 38 wherein the step of comparing comprises performing a whole-brain time-frequency analysis of data from the first and second subsets.

44. An apparatus for assessing a change in central nervous system function, comprising:
a stimuli generator operable to provide a first set of stimuli to a patient to evoke a series of central nervous system responses;
a brain function monitor operable to monitor the patient's brain activity and provide data corresponding to the series of central nervous system responses;
a processor configured to process the data from the brain function monitor to create a first data set representing the patient's responses to the first set of stimuli at a first point in time;
wherein the processor compares the first data set with a second data set representing the patient's responses to stimuli from the stimuli generator at a second point in time and calculates a measurement of central nervous system variation in response to the comparison of the patients first and second data sets, wherein the comparison uses whole-brain assessments in time, frequency or time-frequency domains

45. The apparatus of claim 44 wherein the o stimuli generator is a display for displaying visual stimuli.

46. The apparatus of claim 44 wherein the processor identifies subsets of the first and second data sets corresponding to times that a stimulus is presented to the patient, performs a time-frequency analysis on the subsets of the first and second data sets to transform the data in both time and frequency domains, and compares the transformed subsets of the first data set with the subsets of the second data set to assess the differences between the subsets of the first data set and the subsets of the second data set.

47. The apparatus of claim 44 wherein the processor repeatedly permutes the transformed subsets of the first and second data, sets, wherein for each permutation the transformed subsets are randomly assigned into first and second groups of subsets and the processor compares the first group of subsets with the second group of subsets to assess the differences between the subsets of the first and second groups to generate a distribution of differences based on the comparisons of the permuted subsets of data.

48. The apparatus of claim 47 wherein the processor is configured to generate the measurement of central nervous system variation by comparing the differences assessed from the comparison of the transformed subsets of the first and second data sets with the distribution of differences.

49. The apparatus of any of claim 44 wherein the processor performs the data comparison by performing a whole-brain time-frequency analysis of data from the first and second sets of data.

50. The apparatus of any of claim 44 wherein the brain function monitor comprises an electrode array configured for engaging the patient's scalp.

51. The apparatus of any of claim 44 comprising a sensor for detecting a behavioral characteristic of the patient while the brain function monitors the patient's brain activity wherein the processor calculates a measurement of central nervous system variation in response to the comparison of the patients first and second data sets in combination with a comparison of data from the sensor.

52. The apparatus of claim 51 wherein the sensor examines eye movement of the patient and provides data relating to the eye movement of the patient.
